SEQUENCE LISTING (20 sequences)

SEQ ID NO: 1 - protein, 13 aa, Human immunodeficiency virus
  misc_feature: TAT 48-60
GRKKRRQRRRPPQ

SEQ ID NO: 2 - protein, 13 aa, Human immunodeficiency virus
  misc_feature: R9-Tat
GRRRRRRRRRPPQ

SEQ ID NO: 3 - protein, 17 aa, Human immunodeficiency virus
  misc_feature: HIV-1 Rev 34-50
TRQARRNRRRRWRERQR

SEQ ID NO: 4 - protein, 15 aa, flock house virus
  misc_feature: FHV coat 35-49
RRRRNRTRRNRRRVR

SEQ ID NO: 5 - protein, 19 aa, Brome mosaic virus
  misc_feature: gag 7-25
KMTRAQRRAAARRNRWTAR

SEQ ID NO: 6 - protein, 13 aa, Human T-cell lymphotropic virus type 2
  misc_feature: HTLV-II Rex 4-16
TRRQRTRRARRNR

SEQ ID NO: 7 - protein, 16 aa, Drosophilia atennapedia
  misc_feature: pentratin 43-58
RQIKIWFQNRRMKWKK

SEQ ID NO: 8 - protein, 26 aa, Unknown
  transportan
LIKKALAALAKLNIKLLYGASNLTWG

SEQ ID NO: 9 - protein, 27 aa, Unknown
  transportan
GWTLNSAGYLLGKINLKALAALAKKIL

SEQ ID NO: 10 - protein, 24 aa, Unknown
  transportan
LNSAGYLLGKINLKALAALAKKIL

SEQ ID NO: 11 - protein, 25 aa, Unknown
  transportan
GWTLNSAGYLLGKLKALAALAKKIL

SEQ ID NO: 12 - protein, 21 aa, Unknown
  transportan
AGYLLGKINLKALAALAKKIL

SEQ ID NO: 13 - protein, 22 aa, Unknown
  transportan
LNSAGYLLGKLKALAALAKKIL

SEQ ID NO: 14 - protein, 19 aa, Unknown
  transportan
AGYLLGKLKALAALAKKIL

SEQ ID NO: 15 - protein, 15 aa, Unknown
  transportan
RSRRRRRRSCQTRRR

SEQ ID NO: 16 - DNA, 795 bp, Unknown
  Cre-bla beta-lactamase
atggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 17 - DNA, 19 bp, Artificial
  Sequence derived for suppressing beta-lactamase expression
ccacgatgcctgtagcaat

SEQ ID NO: 18 - RNA, 21 nt, Artificial
  siRNA sequence complementary to portion of IgG heavy chain nucleotide sequence
cgccaagaacuuggucuauuu

SEQ ID NO: 19 - DNA, 23 bp, Artificial
  DNA sequence complementary to IgG heavy chain nucleotide sequence
aacgccaagaacttggtctattt

SEQ ID NO: 20 - RNA, 21 nt, Artificial
  siRNA sequence complementary to portion of IgG heavy chain nucleotide sequence
uugcgguucuugaaccagaua

(19) United States
(12) Patent Application Publication
(10) Pub. No.: US 2005/0255120 A1
(43) Pub. Date: Nov. 17, 2005

(54) Composition and method for introduction of DNA directed RNA interference sequences into targeted cells and tissues

(76) Inventor: Michael R. Simon, Ann Arbor, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/126,562

(22) Filed: May 11, 2005

Related U.S. Application Data
(60) Provisional application No. 60/570,200, filed on May 12, 2004.
(60) Provisional application No. 60/581,474, filed on Jun. 21, 2004.
(60) Provisional application No. 60/605,974, filed on Aug. 31, 2004.
(60) Provisional application No. 60/625,203, filed on Nov. 5, 2004.
(60) Provisional application No. 60/642,317, filed on Jan. 7, 2005.

(51) Int. Cl. A61K 39/395; C12Q 1/68

(52) U.S. Cl. 424/178.1; 435/6; 530/391.1

(57) ABSTRACT

Double-stranded DNA which encodes for a promoter region and for small interfering RNA nucleotide sequences is introduced into specific cells and tissues for the purpose of inhibiting gene expression and protein production in those cells and tissues. Intracellular introduction of the small interfering RNA nucleotide sequences is accomplished by the internalization of a target cell specific ligand bonded to the double-stranded DNA which encodes for a promoter region and for a small interfering RNA nucleotide sequence. The ligand is spontaneously internalized after binding to the cell surface antigen. Optionally, internalization is also facilitated by the binding of a DNA binding protein to the double-stranded DNA. If the unique cell surface antigen is not naturally internalized after binding to its ligand, internalization is promoted by the incorporation of an internalization moiety into the structure of the ligand or attachment of such a peptide to the ligand.

### RELATED APPLICATIONS

This application claims priority of U.S. Provisional Patent Applications Ser. No. 60/570,200 filed May 12, 2004; Ser. No. 60/581,474 filed Jun. 21, 2004; Ser. No. 60/605,974 filed Aug. 31, 2004; Ser. No. 60/625,203 filed Nov. 5, 2004; and Ser. No. 60/642,317 filed Jan. 7, 2005, which are incorporated herein by reference.

### FIELD OF THE INVENTION

The present invention relates in general to gene suppression and in particular to gene suppression through delivery of double-stranded DNA coding for expressible small interfering RNA or short hairpin RNA targeting an over-expressed protein within a subject.

### BACKGROUND OF THE INVENTION

RNA interference (RNAi) is the process whereby messenger RNA (mRNA) is degraded by small interfering RNA (siRNA) derived from double-stranded RNA (dsRNA) containing an identical or very similar nucleotide sequence to that of the target gene. (Waterhouse 2001; Hutvagner and Zamore 2002a and 2002b; Lewis 20020132788; Lewis 20030092180; Kreutzer 20040038921; Scaringe 20040058886). siRNA can be transcribed from DNA templates which have been introduced into living cells (Sui 2002, Bailey 2004). This process prevents the production of the protein encoded by the targeted gene. Allele-specific silencing of dominant disease genes can be accomplished (Miller 2003).

The benefits of preventing specific protein production in mammals include the ability to treat disease caused by such proteins. Such diseases include those that are caused directly by such a protein such as multiple myeloma which is caused by harmful concentrations of a monoclonal immunoglobulin as well as diseases in which the protein plays a contributory role such as the effects of inflammatory cytokines in asthma.

Introduction of dsRNA into mammalian cells induces an interferon response which causes a global inhibition of protein synthesis and cell death. However, dsRNA several hundred base pairs in length have been demonstrated to be able to induce specific gene silencing following cellular introduction by a DNA plasmid (Diallo M et al. Oligonucleotides 2003). Transfer of functional DNA into cells as cargo molecules conjugated to protein ligands has been reported (Sinogeeva et al. 2000, Bogdanova et al. 2001).

### SUMMARY OF THE INVENTION

A composition includes double-stranded DNA (dsDNA) which encodes for a promoter region and for siRNA, or a promoter region and short hairpin RNA (shRNA). The dsDNA is bound to a cell surface receptor specific ligand. The ligand-bound dsDNA is optionally adsorbed to a histone or high mobility group protein that is then internalized by the targeted cell. The dsDNA is then hydrolyzed by a cellular endonuclease thereby releasing dsDNA which is transcribed to siRNA or shRNA. shRNA, if produced, is hydrolyzed by Dicer to release siRNA that silences the target gene. The composition includes a ligand to a receptor or surface antigen that is specific to the target cell. The ligand is a natural peptide, natural protein, or a protein such as an immunoglobulin fragment that is engineered to bind to the targeted receptor. The ligand is bound to dsDNA through bonds such as phosphoramidate, phosphorothioate, or phosphodiester. The internalization of the ligand-bound dsDNA is optionally facilitated by the incorporation of a membrane-permeable arginine-rich peptide, pentratin, transportan, or transportan deletion analogs into the ligand or attachment of such a peptide to the ligand.

### DETAILED DESCRIPTION OF THE PREFERRED EMBODIMENTS

The present invention has utility in suppression of deleterious gene expression products. Production of specific proteins is associated with allergic reactions, transplant organ rejection, cancer, and IgA nephropathy, to name but a few of the medical conditions a subject may suffer. Additionally, according to the present invention, it is appreciated that specific animal proteins are also suppressed in foodstuffs, such as cow's milk, through the treatment of the animal. Inventive compositions include double-stranded DNA (dsDNA) that encodes for a promoter region and for a siRNA (long or short dsRNA), or alternatively, a promoter region and short hairpin RNA (shRNA). The dsDNA is bound to a cell surface receptor specific ligand and is targeted to specific tissues and/or cells upon delivery to a subject. In designing a ligand coupled dsDNA or shRNA, a target tissue and/or cell is selected, and the targeted cell type is analyzed for receptors that internalize ligands following receptor-ligand binding. It is appreciated that the present invention is also operative in suppressing genes within a cell growing in vitro and particularly well suited for limiting contaminants in recombinant protein manufacture.

Cell specific antigens which are not naturally internalized are operative herein by incorporating an arginine-rich peptide within the ligand, an arginine-rich peptide attached to the cell surface receptor specific ligand, as detailed in U.S. Pat. No. 6,692,935 B1 or U.S. Pat. No. 6,294,353 B1. An arginine-rich peptide causes cellular internalization of a coupled molecule upon contact of the arginine-rich peptide with the cell membrane. Pentratin and transportan are appreciated to also be operative as vectors to induce cellular internalization of a coupled molecule through attachment to the cell surface receptor specific ligand as detailed in U.S. Pat. No. 6,692,935 B1 or U.S. Pat. No. 6,294,353 B1.

A cell surface receptor specific ligand as used herein is defined as a molecule that binds to a receptor or cell surface antigen. A ligand is then coupled to an appropriate dsDNA. The ligand is a natural- or engineered-peptide or -protein, such as is commercially available (Antibodies by Design, MorphoSys, Martinsried, Germany). Another specific engineered peptide that is commercially available is the camelid single heavy chain variable domain (Nanobodies, Ablynx, NV; Zwijnaarde, Belgium); such a variable domain heavy chain antibody fragment is humanized and the antigen specificity thereof is generated from a phage display library from an immunized animal (van Koningbruggen et al. 2003) or a nucleic acid sequence expression library from non-immunized animals, as detailed in EP 0 584 421 A1 or U.S. Pat. No. 6,399,763.

If the engineered ligand is an immunoglobulin, the carboxy terminus of the molecule is at the variable end of the protein, and the amino terminus is available for bonding to dsDNA. Because of the relatively large size of immunoglobulin molecules, preferably a Fab fragment is used as the ligand rather than the entire immunoglobin. More preferably, a (Fab′)² fragment is provided that allows for divalent binding as would occur with the entire immunoglobin without the encumbrance of the Fc component. Bridging of cell surface receptors by a divalent (Fab′)² fragment facilitates activation of the signaling pathway and subsequent internalization of the receptor-ligand combination in some internalization processes.

The dsDNA oligonucleotide mediating RNA interference is delivered into the cell by internalization of the receptor. In the event a targeted cell receptor is a unique receptor that is not naturally internalized, that receptor is nonetheless suitable as a target by incorporating an internalization moiety such as an arginine-rich membrane permeable peptide within the ligand or attaching to the ligand: arginine-rich membrane permeable peptide, pentratin, or transportan as detailed in U.S. Pat. No. 6,692,935 B1 or U.S. Pat. No. 6,294,353 B1. This is readily accomplished using established plasmid technology (Caron et al. 2004; He et al. 2004). Alternatively, the use of MorphoSys' commercial trinucleotide mutagenesis technology allows the synthesis of a membrane-permeable arginine-rich peptide at a single position of the variable region, as detailed in U.S. Pat. No. 6,692,935 B1 or U.S. Pat. No. 6,294,353 B1. The MorphoSys system joins an antigen-non-specific Fab fragment containing a membrane-permeable arginine-rich peptide to an engineered Fab fragment with a variable region specific for the cell surface receptor in order to provide for the cell specific targeting of the dsDNA encoding a promoter region and siRNA. These Fab fragments are joined by a helix-turn-helix region. Alternatively, the membrane-permeable arginine-rich peptide is incorporated into the antigen-specific Fab immunoglobulin fragment to yield a bivalent antigen specific molecule produced (Anderson DC 1993). The membrane-permeable arginine-rich peptide is optionally also attached to another portion of the immunoglobulin molecule (Mie M et al. 2003; U.S. Pat. No. 6,692,935 B1; U.S. Pat. No. 6,294,353 B1). Similarly, pentratin or transportan is attached to or incorporated within any ligand portion of the molecule with the proviso that ligand-receptor binding is maintained. In each situation, the ligand containing the membrane-permeable arginine-rich peptide, pentratin, or transportan serves to carry the dsDNA encoding a promoter region and siRNA into the targeted cell.

Arginine-rich peptides which are internalized after contact with the cell membrane have been shown to transport covalently coupled proteins into cells (Peitz M et al. 2002, Jo et al. 2001). Examples of such internalization moieties illustratively include: membrane-permeable arginine-rich peptides, pentratin, transportan and its deletion analogs.

| | |
|---|---|
| GRKKRRQRRRPPQ | (TAT 48-60) |
| | (SEQ ID NO. 1) |
| GRRRRRRRRRPPQ | (R9-TAT) |
| | (SEQ ID NO. 2) |
| TRQARRNRRRRWRERQR | (HIV-1 Rev 34-50) |
| | (SEQ ID NO. 3) |
| RRRRNRTRRNRRRVR | (FHV coat 35-49) |
| | (SEQ ID NO. 4) |
| KMTRAQRRAAARRNRWTAR | (BMVgag7-25) |
| | (SEQ ID NO. 5) |
| TRRQRTRRARRNR | (HTLV-II Rex 4-16) |
| | (SEQ ID NO. 6) |

Other membrane-permeable peptides are pentratin and transportan,

| | |
|---|---|
| RQIKIWFQNRRMKWKK | (Atennapedia |
| | 43-58-pentratin) |
| | (SEQ ID NO. 7) |
| LIKKALAALAKLNIKLLYGASNLTWG. | (transportan) |
| | (Muratovska and |
| | Eccles 2004) |
| | (SEQ ID NO. 8) |

Alternative amino acid composition for transportan and its deletion analogs which maintain membrane transduction properties (Soomets et al. 2000):

| | |
|---|---|
| GWTLNSAGYLLGKINLKALAALAKKIL | (transportan) |
| | (SEQ ID NO. 9) |
| LNSAGYLLGKINLKALAALAKKIL | (transportan7) |
| | (SEQ ID NO. 10) |
| GWTLNSAGYLLGKLKALAALAKKIL | (transportan9) |
| | (SEQ ID NO. 11) |
| AGYLLGKINLKALAALAKKIL | (transportan10) |
| | (SEQ ID NO. 12) |
| LNSAGYLLGKLKALAALAKKIL | (transportan12) |
| | (SEQ ID NO. 13) |
| AGYLLGKLKALAALAKKIL | (transportan14) |
| | (SEQ ID NO. 14) |

TAT=HIV-1 transactivator of transcription; FHV=flock house virus; BMV=brome mosaic virus.

Preferably, the internalization moiety is coupled to an immunoglobin ligand of an inventive dsDNA composition that encodes for a promoter region and small interfering RNA or short hairpin DNA. Nuclear localization of the dsDNA-ligand conjugate is promoted by the presence of TAT (Peitz M et al. 2002).

Receptor-binding immunoglobulins are obtained using hybridoma technology. Fab and (Fab′)² fragments are prepared from such immunoglobulins by papain and pepsin hydrolysis, respectively (Stura et al. 1993). The resulting molecules are purified using standard biochemical methods.

dsDNA which encodes for a promoter region and for siRNA sequences that are complementary to the nucleotide sequence of the target gene are prepared. The siRNA nucleotide sequence is obtained from the siRNA Selection Program, Whitehead Institute for Biomedical Research, Massachusetts Institute of Technology, Cambridge, Mass. (http://jura.wi.mit.edu) after supplying the Accession Number or GI number from the National Center for Biotechnology Information website (www.ncbi.nlm.nih.gov). The Genome Database (www.gdb.org) provides the nucleic acid sequence link which is used as the National Center for Biotechnology Information accession number. Preparation to order of dsDNA, which encodes for the U6 promoter and for siRNA, is commercially available (Promega, Madison, Wis.). Determination of the appropriate sequences is accomplished using the USPHS, NIH genetic sequence data bank. Alternatively, dsRNA containing appropriate siRNA sequences are ascertained using the strategy of Miyagishi and Taira (2003). DsRNA may be up to 800 base pairs long (Diallo M et al. 2003). The dsRNA may have a hairpin structure (U.S. patent Application Publication 2004/0058886). Determination of siRNA sequences optionally is also determined using the Promega algorithm (www.promega.com/sirnadesigner). Invitrogen provides another commercially available RNAi designer algorithm (http://maidesigner.invitrogen.com/maiexpress/).

The ligand and the dsDNA are then coupled with linkages illustratively including phosphoramidate, phosphorothioate, or phosphodiester. Modified dsDNA conjugation to the ligand via a heterobifunctional cross linker (sulfo-succinimidyl 4-(p-maleimidophenyl))-butyrate is accomplished as previously described (Hermanson 1996). Phosphoramidate bonding is accomplished as described (Gryaznov and Winter 1999). Phosphodiester bonding is accomplished using standard biochemical methods. Phosphorothioate bonding can be accomplished as described (Stec et al. 1991). In another embodiment, the dsDNA bonding to the ligand by any of the above described chemical bonds is accomplished via a “helix-turn-helix” region as described in U.S. Pat. No. 5,910,573. This allows maximal flexibility of the dsDNA positioning allowing for successful enzymatic hydrolysis. Endonucleases most readily cleave phosphodiester bonds (Evans and Aguilera 2003).

In an additional embodiment, a disulfide linkage between the dsDNA and ligand is formed (Hermanson 1996). The dsDNA is modified with a cystamine at the 5′ phosphate group using a carbodiimide reaction such as reaction with 1-ethyl-3-(3-dimethylaminopropyl)carbodiimide HCl (Hermanson pages 651-654). Reaction with a disulfide reducing agent creates a thiol group. The ligand is modified with a heterobifunctional cross linker illustratively N-succinimidyl 3-(2-pyridyldithio)propionate (Hermanson pages 230-232). Disulfide linkage between the cystamine-modified dsDNA and the antibody ligand modified with the heterobifunctional cross linker is accomplished as described for enzyme conjugation to DNA (Hermanson pages 662-664). Additionally, dsDNA ligand conjugation is also via stable hydrazone bonds (Solulink BioConjugation System, Solulink, San Diego, Calif.; Kozlov et al. 2004).

The suitability of the resulting ligand-dsDNA as a substrate for endonuclease is determined in vitro with optimal ligand molecule size and dsDNA length being determined. Additionally, the necessity and performance of an internalization moiety is determined in vitro.

In an alternate embodiment, the ligand-dsDNA molecule is adsorbed onto a DNA binding molecule(s), illustratively including a histone, a high mobility group (nonhistone chromosomal) protein (HMG) or protamine (Brewer et al.; Bianchi et al. 1994). Histones with relatively greater DNA-histone dissociation constants such as histone H3 and histone H4 (Upstate Biotechnology, Lake Placid, N.Y.) are preferred. While not being bound by a particular theory, it is believed adsorption of an inventive composition is via multiple hydrogen bonds. Alternatively, HMG1 or MHG2 (prepared as described by Gaillard and Strauss, 1994) which have even lower binding affinity are operative herein. Lower binding affinity of the adsorbed molecule is preferred to facilitate dissociation of the ligand-dsDNA-histone complex or ligand-dsDNA-HMG complex following cellular internalization.

Protamines are arginine-rich proteins. For example, protamine 1 contains 10 arginine residues between amino acid residue number 21 and residue number 35 (RSRRRRRRSCQTRRR) (Lee et al. 1987) (SEQ ID NO. 15). Protamine binds to DNA (Bianchi et al. 1994; Brewer et al. 2003).

Preparation of the ligand-DNA-histone complex is accomplished as described by (Yoshikawa et al. 2001). Complexes of lysine rich histone containing 24.7% (w/w) lysine and 1.9% arginine (w/w) with DNA is prepared by gentle dilution from a 2 M NaCl solution. Histone and DNA are dissolved in 2 M NaCl/10 mM Tris/HCl, pH 7.4, in which the charge ratio of DNA:histone (±) is adjusted to 1.0. Then the 2 M NaCl solution is slowly dispersed in distilled water in a glass vessel to obtain 0.2 M and 50 mM NaCl solutions. The final volume is 200 μL and final DNA concentration is 0.75 μM in nucleotide units. Binding affinity of histones is further reduced by acetylation of the lysine residues of the histone. This reduces the positive charge of the ε-NH₂ group. Acetylation is accomplished by established techniques (Garcea and Alberts 1980). Preparation of the ligand-dsDNA-HMG complex is accomplished using the method of (Yaneva et al. 1997). HMG and DNA are dissolved in 10 mM Tris/HCl, pH 7.8, 20 mM NaCl, 0.1 mM phenylmethylsulfonyl fluoride. Alternatively, the method of (Guillard and Strauss 2000) is used in which the HMG and DNA are incubated in 25 mM Tris/HCl, pH 7.5, 50 mM NaCl, 1 mM EDTA and 1 mM dithiothreitol.

Preparation of the ligand-dsDNA-protamine complex is accomplished as described by (Bianchi et al. 1994). The protamine (human recombinant protamine 1, Abnova Corporation, Taiwan, www.abnova.com.tw) and ligand-dsDNA at a protamine arginine to oligonucleotide phosphate ratio of 0.1 are placed in a buffered solution containing 10% glycerol (v/v), 10 mM Tris-HCl, 5 mM MgCl₂ and 50 mM NaCl at pH 7.5. The solution is incubated at room temperature for 1 hr.

The constructed ligand-dsDNA molecule is then administered parenterally and binds to its target cell via its receptor. The constructed ligand-dsDNA molecule is then internalized and the dsDNA is hydrolyzed by endonuclease thereby releasing dsDNA which encodes for a promoter region and for siRNA for gene silencing.

### EXAMPLE 1

The Invitrogen Corporation (Carlsbad, Calif.) CellSensor CRE-bla Jurkat Cell-based Assay is used. The detailed protocol is available online and is included in the references (CellSensor protocol). Jurkat cells express CD38 on their cell surfaces which is internalized following ligand binding to it (Funaro at al. 1998). CellSensor CRE-bla Jurkat Cell-based Assay contains a beta-lactamase reporter gene under control of a cAMP response element which has been stably integrated into the CRE-bla Jurkat cell line (clone E6-1). Beta-lactamase is expressed following forskolin stimulation.

Double-stranded DNA encoding for a promoter region and for short interfering RNA 19 base pairs long is prepared using the Invitrogen Corporation algorithm based on the DNA sequence of the CRE-bla beta-lactamase gene:

| | |
|---|---|
| atggacccagaaacgctggtgaaagtaaaagatg | (SEQ ID NO. 16) |
| ctgaagatcagttgggtgcacgagtgggttacat | |
| cgaactggatctcaacagcggtaagatccttgag | |
| agttttcgccccgaagaacgttttccaatgatga | |
| gcacttttaaagttctgctatgtggcgcggtatt | |
| atcccgtattgacgccgggcaagagcaactcggt | |
| cgccgcatacactattctcagaatgacttggttg | |
| agtactcaccagtcacagaaaagcatcttacgga | |
| tggcatgacagtaagagaattatgcagtgctgcc | |
| ataaccatgagtgataacactgcggccaacttac | |
| ttctgacaacgatcggaggaccgaaggagctaac | |
| cgcttttttgcacaacatgggggatcatgtaact | |
| cgccttgatcgttgggaaccggagctgaatgaag | |
| ccataccaaacgacgagcgtgacaccacgatgcc | |
| tgtagcaatggcaacaacgttgcgcaaactatta | |
| actggcgaactacttactctagcttcccggcaac | |
| aattaatagactggatggaggcggataaagttgc | |
| aggaccacttctgcgctcggcccttccggctggc | |
| tggtttattgctgataaatctggagccggtgagc | |
| gtgggtctcgcggtatcattgcagcactggggcc | |
| agatggtaagccctcccgtatcgtagttatctac | |
| acgacggggagtcaggcaactatggatgaacgaa | |
| atagacagatcgctgagataggtgcctcactgat | |
| taagcattggtaa. | |

The DNA nucleotide sequence derived for suppressing beta-lactamase synthesis is: CCACGATGCCTGTAGCAAT (SEQ ID NO. 17). The dsDNA encoding for a promoter region and for the complementary RNA oligonucleotide is prepared and modified to contain a 3′ amino group with a 3 carbon linker (3′N3). Modified dsDNA which encodes for a promoter region and for the siRNA and which contains the 3′N3 is formed by annealing it with its complementary strand sequences. This double-stranded DNA encoding for a promoter region and for siRNA is then incubated with a heterobifunctional crosslinker, sulfosuccinimidyl 4-(p-maleimidophenyl)-butyrate as described by (Chiu et al. 2004). The product is then conjugated (Hermanson 1996) to anti-CD38 antibody (Serotec, Raleigh, N.C.) and to the anti-CD38 (Fab′)² fragment via a cysteine residue added to the amino terminal of the antibody. Oligonucleotide-protein conjugation is commercially available (SoluLink, San Diego, Calif.). The dsDNA-antiCD38 conjugate is incubated at 37° C. with the Jurkat cells for from 4 to 24 hours at concentrations ranging from 100 pM to 200 nM to evaluate efficacy. Typical efficacy is at 2 nM. Effective knockdown of intracellular synthesis of beta-lactamase is demonstrated in this system by the appearance of green cellular fluorescence. Positive control cells which produce beta-lactamase fluoresce blue.

### EXAMPLE 2

Multiple myeloma is a fatal incurable disease caused by the production of large amounts of a monoclonal immunoglobulin by malignant plasma cells (Grethlein S, Multiple Myeloma, eMedicine 2003). CD38 is a cell surface receptor found on myeloma plasma cells (Almeida J et al. 1999). Ligation of CD38 with anti-CD38 monoclonal antibodies ((Serotec, Raleigh, N.C. and others)) results in CD38 internalization (Pfister et al. 2001).

Anti-CD38 monoclonal antibodies are hydrolyzed by pepsin to produce anti-CD38 (Fab′)² fragments. The anti-CD38 (Fab′)² fragments are then conjugated by phosphodiester linkage to dsDNA which encodes for a promoter region and for an siRNA sequence that is complementary to a portion of the nucleotide sequence of the rearranged heavy chain of IgG. In this case the nucleotide sequence link is X98954 and the GI number is 1495616. The siRNA sequences provided by the Whitehead Institute are:

| | | |
|---|---|---|
| S 5′: | CGCCAAGAACUUGGUCUAU UU | (SEQ ID NO. 18) |
| cDNA: | AA CGCCAAGAACTTGGTCTAT TT | (SEQ ID NO. 19) |
| AS 3′: | UU GCGGUUCUUGAACCAGAUA | (SEQ ID NO. 20) |

Alternatively, the anti-CD38 monoclonal antibodies are conjugated to the dsDNA containing a siRNA sequence that is complementary to a portion of the nucleotide sequence of the rearranged heavy chain of the IgG subclass of the subject's monoclonal IgG, i.e., IgG₁, IgG₂, IgG₃ or IgG₄.

Effective doses of anti-CD38(Fab′)²-dsDNA need to be administered at intervals ranging from one day to several days in order to maintain suppression of IgG production. Because the half life of IgG is approximately 23 days, the circulating concentration of the myeloma IgG will decrease gradually over several months. Suppression of the IgG subclass to which the IgG myeloma protein belongs allows maintenance of IgG mediated immunity because the remaining IgG subclasses are not reduced. Improvement and/or prevention of aspects of the disease which are consequences of high concentrations of the myeloma protein occur gradually as the concentration of the myeloma protein decreases. A direct effect of high concentrations of myeloma protein is hyperviscosity. This morbid effect of multiple myeloma is inhibited. Varying doses of the dsDNA-ligand conjugate ranging from 0.4 to 15 grams of the anti-CD38 (Fab′)²-dsRNA are administered depending upon response.

The anti-CD38 (Fab′)²-long dsDNA which encodes for a promoter region and for the above-described siRNA then binds to CD38 on the surfaces of the subject's plasma cells. Following internalization, endonuclease hydrolyses the dsDNA-ligand conjugate. shRNA is transcribed. Dicer hydrolyzes the shRNA into siRNA which then interrupts the malignant plasma cell production of the IgG myeloma protein.

Since allele-specific silencing of dominant disease genes can be accomplished (Miller 2003), selective interruption of the production of the monoclonal IgG while allowing production of normal IgG antibodies is accomplished.

### EXAMPLE 3

Allergic disease is mediated via IgE binding to the surfaces of mast cells and basophils. Upon bridging of adjacent IgE molecules by antigen, the mast cells and basophils are activated and release their mediators (Siraganian 1998). IgE binding by mast cells and basophils causes the signs and symptoms of allergic rhinitis, asthma, food and drug allergy, and anaphylaxis (e.g. Becker 2004). The amino acid sequence of the CH3 region of human IgE is available as are many of the codons (Kabat E A 1991). The DNA nucleotide sequence of the CH3 region of human IgE is readily deduced. The deduced CH3 region sequence is then provided to the Whitehead Institute's internet site as above to yield the corresponding siRNA sequence.

The anti-CD38 (Fab′)²-dsDNA which encodes for a promoter region and for the anti-IgE siRNA then binds to CD38 on the surfaces of the subject's plasma cells. Following internalization, endonuclease hydrolyses the dsDNA-ligand conjugate. shRNA is transcribed and Dicer hydrolyzes the shRNA into siRNA which then interrupts the plasma cells' production of the IgE. Over several months the mast cell-bound and basophil-bound IgE is released and metabolized. The mast cell and basophil IgE receptors decrease markedly and the subject loses allergic reactivity.

### EXAMPLE 4

IgA nephropathy is an incurable disease of the kidney caused by deposition of IgA in the glomeruli of the kidneys (Brake M 2003). IgA₁ or IgA₂ production is interrupted, depending upon the IgA subclass in the glomeruli, as described above for the silencing of IgG production. The progressive kidney damage caused by IgA is thereby interrupted.

### REFERENCES

Almeida J, Orfao A, Mateo G, Ocqueteau M, Garcia-Sanz R, Moro M J, Hernandez J, Ortega F, Borrego D, Barez A, Mejida M, San Miguel J F. Immunophenotypic and DNA content characteristics of plasma cells in multiple myeloma and monoclonal gammopathy of undetermined significance. Path Biol 1999; 47:119-127.
Anderson D C, Nichols E, Manger R, Woodle D, Barry M, Fritzberg A R. Tumor cell retention of antibody Fab fragments is enhanced by an attached HIV TAT protein-derived peptide. Biochem Biophys Res Commun 1993; 194:876-884.
Bailey C, Betz, N, Sprecher C, Storts D, Vidugiriene J, Yeager T. DNA-directed RNA interference: hairpin cloning and expression made easy. Promega Notes, number 87, pp 7-10, 2004. www.promega.com.
Becker J M. Allergic Rhinitis, in In eMedicine, eds: Park C L, Mary L Windle M L, Georgitis J W, Pallares D, M D, Ballow M. 2004.
Bianchi F, Rousseaux-Prevost R, Bailly C, Rousseaux J. Interaction of human P1 and P2 protamines with DNA. Biochem Biophys Research Commun 1994; 201:1197-1204.
Bogdanova M A, Sinogeeva N, Akifiev B N, Zamiralova E, Perevozchikov A, Baranov V, Shavlovsky M. P1637 Developing of non-viral system for transfer foreign DNA. (abstract) Vienna Academy of Postgraduate Medical Education and Research 2001.
Brake M, Somers D. IgA Nephropathy in eMedicine, eds: Sondheimer J H, Talavera, F, Thomas C, Schmidt R J, Vecihi Batuman V. 2003.
Brewer L, Corzett M, Lau E Y, Balhom R. Dynamics of protamine 1 binding to single DNA molecules. J Biol Chem 2003; 43:42403-42408.
Caron N J, Quenneville S P, Tremblay J P. Endosome disruption enhances functional nuclear delivery of Tat-fusion proteins. Biochem Biophys Res Commun 2004; 319:12-20.
CellSensor CRE-bla Jurkat Cell-based Assay Protocol, Catalogue number K1134 (K1079), Invitrogen Corporation, Carlsbad, Calif.
Diallo M, Arenz C, Schmitz K, Sandhoff K, Scheppers U. Long endogenous dsRNAs can induce complete gene silencing in mammalian cells and primary cultures. Oligonucleotides 2003; 13:381-392.
Evans C J, Aguilera R J. Dnase II: genes, enzymes and function. Gene 2003; 322:1-15.
Funaro A, Reinis M, Trubiani O, Santi S, Di Primio R, Malavasi F. CD38 functions are regulated through an internalization step. J Immunol 1998; 160:2238-2247.
Futaki S, Goto S, Sugiura Y. Membrane permeability commonly shared among arginine-rich peptides. J Mol Recognit 2003; 16:260-264.
Gaillard C, Strauss F. Association of poly(CA).poly(TG) DNA fragments into four-stranded complexes bound by HMG1 and 2. Science 1994; 264(5157):433-6.
Gaillard C, Strauss F. High affinity binding of proteins HMG1 and HMG2 to semicatenated DNA loops. BMC Molecular Biology 2000: 1; 1 Epub Oct. 18, 2000.
Garcea R L, Alberts B M. Comparative studies of hi stone acetylation in nucleosomes, nuclei, and intact cells. Evidence for special factors which modify acetylase action. J Biol Chem 1980; 255:11454-11463
Grethlein S. Multiple Myeloma. In eMedicine, eds: Krishnan K, Talavera F, Guthrie T H, McKenna Rajalaxmi, Besa EC 2003.
Gryaznov S M, Winter H. RNA mimetics: oligoribonucleotide N3′→P5′ phosphoramidates. Nucl Acids Res 1999; 26:4160-4167.
He D, Yang H, Lin Q, Huang H. Arg9-peptide facilitates the internalization of an anti-CEA immunotoxin and potentiates its specific cytotoxity to target cells. Int J Biochem Cell Biol 2004; 37:192-205.
Hermanson G T. Bioconjugate Techniques. Academic Press, San Diego, Calif. 1996.
Hutvagner G, Zamore P D. A microRNA in a multiple-turnover RNAi enzyme complex. Science 2002; 297:2056-2060.
Hutvagner G, Zamore P D. RNAi: nature abhors a double-strand. Curr Opinion in Genetics and Development 2002; 12:225-232.
Jo D, Nashabi A, Doxcee C, Lin Q, Unutmaz D, Chen J, Ruley H E. Epigenetic regulation of gene structure and function with a cell-permeable Cre recombinase. Nature Biotechnology 2001; 19:929-933.
Kabat E A, Wu T T, Perry H M, Gottesman K S, Foeller C. Sequences of Proteins of Immunological Interest. Fifth Edition. Tabulation and Analysis of Amino Acid and Nucleic Acid Sequences of Precursors, V-Regions, C-Regions, J-Chain, T-Cell Receptors for Antigen, T-Cell Surface Antigens, β₂-Microglobulins, Major Histocompatibility Antigens, Thy-1, Complement, C-Reactive Protein, Thymopoietin, Integrins, Post-gamma Globulin, α₂-Macroglobulins, and other Related Proteins. 1991. NIH Publication Number 91-3242.
Kozlov I A, Melnyk P C, Stromborg K E, Chee M S, Barker D L, Zhao C. Efficient strategies for the conjugation of oligonucleotides to antibodies enabling highly sensitive protein detection. Biopolymers 2004; 73:621-630.
Lee C-H, Hoyer-Fender S, Engel W. The nucleotide sequence of a human protamine 1 cDNA. Nucleic Acids Research 1987; 15:7639.
Mie M, Takahashi F, Funabashi H, Yanagida Y, Aizawa M, Kobatake E. Intracellular delivery of antibodies using TAT fusion protein A. Biochem Biophys Res Commun 2003; 310:730-734.
Miller V M, xia H, Marrs G L, Gouvion C M, Lee G, Davidson B L, Paulson H L. Allele-specific silencing of dominant disease genes. Proc Natl Acad Sci USA 2003; 100:7195-7200.
Miyagishi M, Taira K. strategies for generation of an siRNA expression library directed against the human genome. Oligonucleotides 2003; 13:325-333.
Peitz M, Pfannkuche K, Rajewsky K, Edenhofer F. Ability of the hydrophobic FGF and basic TAT peptides to promote cellular uptake of the recombinant Cre recombinase: A tool for efficient genetic engineering of mammalian genomes. Proc Natl Acad Sci USAS 2002; 99:4489-4494.
Pfister M, Ogilvie A, da Silva C P, Grahnert A, Guse A H, Hauschildt S. NAD degradation and regulation of CD38 expression by human monocytes/macrophages. Eur J Biochem 2001; 268:5601-5608.
Sinogeeva N I, Bogdanova M A, Aleinikova T D, Shavlovskii M M, Gaitskhoki V S, Ostapenko O V, Glazkov P B, Baranov V S. Expression of marker genes in muscle fibers after transfection in vivo, mediated by lactoferrin. Genetika 2000; 36:749-757.
Siraganian R P. Biochemical events in basophil or mast cell activation and mediator release. Chapter 16 pp 204-227 in Allergy Principles and Practice, 5^{th} edition, eds E Middleton, Jr, C E Reed, E F Ellis, N F Adkinson, Jr, J W Yunginger, W W Busse. Mosby, St. Louis, 1998).
Soomets U, Lindgren M, Gallet X, Hallbrink M, Elmquist A, Balaspiri L, Zorka M, Pooga M, Brasseur R, Langel U. Deletion analogues of transportan. Biochem Biophys Acta 2000; 1467:165-176.
Stec W J, Grajkowski A, Kozioliewiecz M, Uznanski B. Novel route to oligo(deoxyribonucleoside phosporothioates). Stereocontrolled synthesis of P-chiral oligo(deoxyribonucleoside phosporothioates). Nucl Acids Res 1991; 19:5883-5888.
Stura E A, Fieser G G, Wilson I A. Crystallization of antibodies and antibody-antigen complexes. Immunomethods 1993; 3:164-179.
Sui G, Soohoo C, Affar E B, Gay F, Shi Y, Forrester W C, Shi Y. A DNA vector-based technology to suppress gene expression in mammalian cells. Proc Natl Acad Sci USA 2002; 99:5515-5520.
van Koningsbruggen S, de Haard H, de Kievit P, Dirks R W, van Remoortere A, Groot A J, van Engelen B G, den Dunnen J T, Verrips C T, Frants R R, van der Maarel S M. Llama-derived phage display antibodies in the dissection of the human disease oculopharyngeal muscular dystrophy. J Immunol Methods 2003; 279: 149-161.
Waterhouse P M, Wang M-B, Lough T. Gene silencing as an adaptive defense against viruses. Nature 2001; 411:834-842.
Yaneva J, Leuba S H, van Holde K, Zlatanova J. The major chromatin protein histone H1 binds preferentially to cis-platinum-damaged DNA. Proc Natl Acad Sci USA 1997; 94:13448-13451.
Yoshikawa Y, Velichko Y S, Ichiba Y, Yoshikawa K. Self-assembled pearling structure of long duplex DNA with histone H1. Eur J Biochem 2001; 268:2593-2599.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A composition comprising: a cell surface receptor specific ligand having a bond to a double-stranded DNA, said double-stranded DNA encoding for a promoter region functionally linked to express small interfering RNA or short hairpin RNA operative to suppress production of a cellular protein.

2. The composition of claim 1 wherein said ligand is a protein.

3. The composition of claim 2 wherein said protein is synthetic.

4. The composition of claim 1 wherein said ligand is an immunoglobulin.

5. The composition of claim 4 wherein said bond is between an amino terminus of said double-stranded DNA.

6. The composition of claim 2 wherein said protein is a Fab fragment of an immunoglobulin molecule.

7. The composition of claim 2 wherein said protein is a (Fab′)² immunoglobulin fragment.

8. The composition of claim 1 wherein said small interfering RNA is complementary to a cellular nucleotide sequence for a cell binding said ligand.

9. The composition of claim 1 wherein the bond between said ligand and said double-stranded DNA is selected from the group consisting of: disulfide bond, heterobifunctional crosslinker bond, hydrazone bond, phosphoramidate bond, phosphorothioate bond, and phosphodiester bond.

10. The composition of claim 1 wherein the bond is in a helix-turn-helix region.

11. The composition of claim 1 further comprising a double-stranded DNA binding molecule adsorbed to said double-stranded DNA.

12. The composition of claim 12 wherein said double-stranded DNA binding molecule is selected from the group consisting of: a histone, a protamine, and a high mobility group protein.

13. The composition of claim 1 further comprising an internalization moiety operative as a cellular membrane transporter.

14. The composition of claim 1 wherein said cell surface receptor specific ligand is anti-CD38 (Fab′)² and said double-stranded DNA is complementary to a portion of a malignant cell genome.

15. The composition of claim 1 wherein said cell surface receptor specific ligand is anti-CD38 (Fab′)² and said double-stranded DNA encodes an anti-immunoglobulin small interfering RNA.

16. The composition of claim 16 wherein said anti-immunoglobulin small interfering RNA is selected from the group consisting of: anti-IgA, anti-IgG, anti-IgE, and anti-IgM small interfering RNA.

17. A composition comprising: a cell surface receptor specific component ligand having an internalization moiety coupled to or incorporated into said ligand, additionally having a bond to a double-stranded DNA encoding a small interfering RNA or a small hairpin RNA sequence operative to suppress production of a cellular protein and encoding a promotor region and said internalization moiety is selected from the group consisting of: an arginine-rich peptide, pentratin, transportan and transportan deletion analogs.

18. A process for suppressing cellular production of a protein comprising: exposing a cell having a cell surface receptor to a composition of claim 1 wherein said ligand binds to said receptor and said double-stranded DNA is complementary to a cellular nucleotide gene sequence implicated in the cellular production of the protein.

19. The process of claim 19 wherein expression of the protein is associated with a medical condition.

20. The process of claim 19 wherein said cell is in vivo.
